(19) United States
(12) Patent Application Publication
(10) Pub. No.: US 2022/0331623 A1
(43) Pub. Date: Oct. 20, 2022

(54) METHODS AND SYSTEMS ASSOCIATED WITH CONTROLLING AIR FLOW WITHIN A MASK

(71) Applicant: HONG WANG, CYPRESS, TX (US)

(72) Inventor: HONG WANG, CYPRESS, TX (US)

(21) Appl. No.: 17/685,560

(22) Filed: Mar. 3, 2022

Related U.S. Application Data
(60) Provisional application No. 63/176,437, filed on Apr. 19, 2021.

(51) Int. Cl. A62B 23/02; A41D 13/11; A41D 31/14; A62B 9/02

(52) U.S. Cl. CPC A62B 23/025; A41D 13/1161; A41D 31/145; A62B 9/02

(57) ABSTRACT

A mask with a seal along an upper portion of a mask to divert air flow along a lateral axis of the mask.

### BACKGROUND INFORMATION

### Field of the Disclosure

Examples of the present disclosure relate to systems and methods for controlling air flow within a mask. More specifically, embodiments relate to passive systems that are configured to form a seal along an upper portion of a mask to divert air flow along a lateral axis of the mask below the seal.

### Background

Masks are face coverings that are worn over the mouth and nose of a user. Masks create a physical barrier separating the mouth and nose of the user from potential contaminants in the immediate environment. If worn properly, masks help block large-particle droplets that may contain virus and bacteria from reaching the user's mouth and nose. As such, masks help reduce exposure to respiratory secretions.

Conventional masks are particulate-filtering faceplates or filtering surfaces having straps that are worn over a user's ears attempting to form complete seals around a user's face. However, typically there may be a gap naturally formed between the inner surface of the filtering surface and the user's face around the nose ridge area. Due to the existence of this gap, masks do not divert airflow from the user away from the user's eyes, which may cause glasses to fog. To minimize this gap, mask has incorporated a nose strip formed by a metal wire to enforce the fitting of a mask to a face. However, the fitting is often loose allowing air to be easily leaked through the gap. Alternatively, particulate-filtering faceplates form full seals around a user's face, which due to this complete seal there is no passive control of air flow laterally from a user's nose or mouth.

Accordingly, needs exist for system and methods for controlling air flow within a mask.

### SUMMARY

Examples of the present disclosure relate to systems and methods for a mask, face covering, etc. that is configured to passively control air flow. Based on an inner profile of the mask air is passively diverted along a lateral axis of the mask away from the user's nose and mouth. In embodiments, the mask may include ear straps, filtering surface, and inflatable bag.

The ear straps may be positioned on sidewalls of the mask, and form loops on the outer edges of the filtering surface. The ear straps may have a first end that is positioned proximal to an upper portion of the outer edge, and a second end that is positioned proximal to a lower portion of the outer edge. The ear straps may be configured to be coupled to ears of a user to retain the mask on the user's face.

The filtering surface may be a mechanical barrier that interferes with the direct airflow in and out of respiratory orifices of a user. The filtering surface may be configured to limit particles from moving across the front surface and the rear surface of the mask. In embodiments, the filtering surface may include multiple layers of various materials that filter aerosol particles.

The inflatable bag may be a container, balloon, plastic bag, receptacle, etc. that is configured to increase in thickness based on a volume of air positioned within the inflatable bag. Therefore, the inflatable bag may be made of flexible and airtight materials. The inflatable bag may be configured to extend along a lateral axis of the filtering surface, and may have a first end positioned proximate to a first outer edge of the filtering surface and a second end positioned proximate to a second outer edge of the filtering surface.

Theoretically, assuming no gravity, an empty inflatable bag made of a totally flexible material may start to be inflated when fluid, such as air or water, is injected into the inflatable bag. The inflatable bag may contain a maximum volume of incompressible fluid, such as water, which is positioned within the bag without stretching the bag material. This volume is the bag volume. When the bag is full or close to full, a gentle squeeze on the bag may increase the bag pressure and harden the bag substantially.

If the bag material is very elastic such as latex and therefore very stretchable, continuing the injection of fluid within the bag may inflate the bag to a much larger volume than the bag volume and the bag pressure may continue to build up. If the bag material is non-elastic such as plastic film and therefore not much stretchable, continuing the injection of fluid may not substantially further inflate the bag but the bag pressure may build up quicker and eventually may cause the bag to burst. So an inflatable bag with fluid volume close to its bag volume may be hardened and show the shape of the bag. However, if the fluid within the inflatable bag is substantially less than full, the inflatable bag may be very flexible and a gentle squeeze on the bag may not affect its pressure at all.

For example, if an empty plastic bag is substantially larger than a cup, filling the bag with a cup of water, and then placing the bag with water into the cup, may cause the water within the bag to conform with the shape of the cup and fill up the cup as if there were no bag at all. Due to the existence of the bag, if more than a cup of water is put in the bag, the water that exceeds a cup volume may simply be held by a portion of the bag positioned outside and above the cup. If there is less water in the bag than the cup volume, the water within the bag may still conform with the cup shape covering the entire bottom of the cup without filling the whole cup. The above may be true even when the fluid is air. Though air is very compressive, a certain quantity of air may still have a specific volume at a certain temperature and pressure, such as ambient temperature and pressure.

The space defined by the gap between the inner surface of a filtering surface and a user's face around the nose ridge has a gap volume. When the inflatable bag, which is larger, especially much larger, than the gap volume, is filled with air, the inflatable bag preferably has a volume close to the gap volume. The inflatable bag is therefore only partially inflated or even substantially partially inflated and may be very flexible and easy to be shaped to thicken enough in the gap, where breath-out air may leak through. Any excess air within the inflatable bag that is greater than the gap volume needed to seal the space, if any in the bag, may simply be contained in other areas of the inflatable bag, which may extend downward below the gap. These portions of the inflatable bag extending downward from the gap may not affect the sealing above or at the gap. If the air within the inflatable bag has a volume less than the gap volume and the air within the inflatable bag may not thicken all the way from top to bottom inside the gap, it may still thicken enough in the top part to stop the leakage. In this way, a mask with one configuration of the inflatable bag may fit a variety of faces and block the air emitted from the user's nose and mouth. Further, a user may utilize their hands to push the bag when the mask is worn, which may help to thicken the inflatable bag at desired locations and optimize the seal.

The inflatable bag may have a bag volume larger or even substantially larger than a gap volume. The inflatable bag may be configured to be inflated or prefilled with air with a volume preferably but not necessarily equal to the gap volume. In this case, the inflatable bag may be only partially inflated or even substantially partially inflated and may be very flexible. By partially inflating the bag with air and positioning the bag along an upper and inner surface of the filtering surface, a varying profile of the inner surface of the mask may be created by increasing a thickness of the mask at desirable locations, confined by the filtering surface and its user's face. This may divert aerosol particles emitted from the user's nose and mouth to the first outer edge and the second outer edge of the filtering surface, restricting or limiting the aerosol particles from interacting with a user's eyes or glasses, reducing fogging of glasses.

The inflatable bag, which has a bag volume larger than a gap volume, may be configured to be inflated after the mask is put on with air of a volume enough to fill the gap, preferably equal to or less than the gap volume. In this case, the inflatable bag, which has a bag volume larger than the gap volume, may be only partially inflated or even substantially partially inflated and may be very flexible. When the inflatable bag is only partially inflated or substantially inflated, the bag may have a shape much different from the shape when it is fully inflated. With only partial inflation, the bag may be easily shaped according to its confinement. In this way, one configuration of the inflatable bag may fit and seal the gap for many different faces.

These, and other, aspects of the invention will be better appreciated and understood when considered in conjunction with the following description and the accompanying drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 depicts a mask to passively control airflow, according to an embodiment.

FIG. 2 depicts a mask to passively control airflow, according to an embodiment.

FIG. 3 depicts a mask to passively control airflow, according to an embodiment.

FIG. 4 depicts a method associated with mask to passively control airflow, according to an embodiment.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION

In the following description, numerous specific details are set forth in order to provide a thorough understanding of the present embodiments. It will be apparent, however, to one having ordinary skill in the art, that the specific detail need not be employed to practice the present embodiments. In other instances, well-known materials or methods have not been described in detail in order to avoid obscuring the present embodiments.

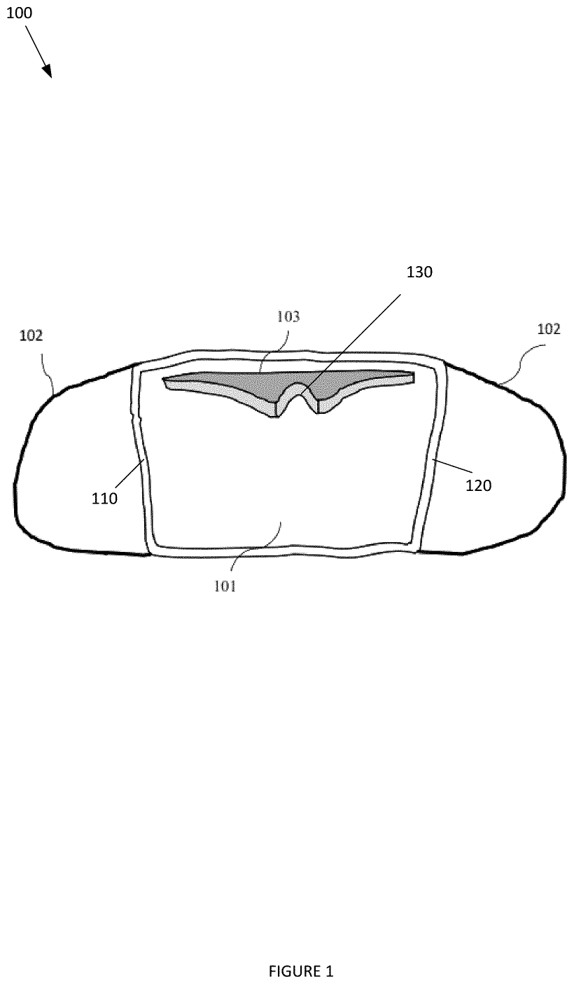

FIG. 1 depicts a mask 100 configured to passively control air flow of particles emitted from a user, according to an embodiment. Mask 100 may include ear straps 102, filtering surface 101, and an inflatable bag 103 that has a bag volume preferably larger than a gap volume.

Ear straps 102 may be positioned on sidewalls of the mask 100. Ear straps 102 form loops on the outer edges of the filtering surface 101, wherein a lateral axis of mask 100 extends from first outer edge 110 to second outer edge 120. Ear straps 102 may have a first end that is positioned proximal to an upper portion of the outer edge 110, and a second end that is positioned proximal to a lower portion of the outer edge 110. Ear straps 102 may be configured to be coupled to ears of a user to retain the mask 100 on the user's face. In other embodiments, other mechanisms or devices may be utilized to secure mask 100 to the user's face.

Filtering surface 101 may be a mechanical barrier that interferes with the direct airflow in and out of respiratory orifices of the user. Filtering surface 101 may be configured to limit particles from moving across the front surface and the rear surface of the mask. In embodiments, filtering surface 101 may include multiple layers of various materials that filter aerosol particles.

Inflatable bag 103 may be a container, balloon, plastic bag, receptacle, etc. that is configured to increase in thickness based on a volume of air positioned within inflatable bag 103. Inflatable bag 103 may be configured to extend along the lateral axis of the filtering surface 101 below an upper edge of filter surface 101, and may have a first end positioned proximate to first outer edge 110 of the filtering surface 101 and a second end positioned proximate to second outer edge 120 of the filtering surface 101. The inflatable bag 103 may have varying profiles according to the shapes of the gap between the inner surface of the filtering surface and a typical user's face around the nose ridge. The inflatable bag 103 may have a bag volume larger than a gap volume. The inflatable bag 103 may have a bag volume substantially larger than a gap volume. Inflatable bag 103 may be configured to be partially inflated and/or prefilled with air. By partially inflating the bag with air and positioning the bag along an upper and inner surface of the filtering surface, a varying profile of the inner surface of the mask may be created by increasing a thickness of the mask 100 at desirable locations.

In embodiments, partial inflation means the inflatable bag 103 is filled with air appreciably less than a bag volume under the ambient pressure, wherein the pressure within the inflatable bag 103 is substantially similar to that of the ambient air pressure. When the inflatable bag 103 is partially inflated, the inflatable bag 103 is not stretched much by the air within inflatable bag 103. If the inflatable bag 103 is filled with air at a volume equal to the bag volume, the inflatable bag 103 is fully filled and the pressure within the inflatable bag 103 may be still substantially similar to that of the ambient air pressure. However, such a fully infilled or inflated bag may be at the verge of being stretched and hardened. When the fully inflated bag is inflated further by injecting more air, the bag may start to harden by the bag pressure building up, and having a pressure that may be greater than the ambient air pressure. A bag volume can be approximately measured by filling up the inflatable bag 103 with incompressible fluid, such as water. In embodiments, inflatable bag 103 may be partially inflated when inflatable bag 103 is filled with approximately 1% to 95% of the bag volume at ambient pressure. When inflatable bag 103 is partially inflated it is more flexible, pliable, and more adaptable to conform with a confined space, such as the gaps between the inner surface of a filtering surface and a user's face.

In embodiments, inflatable bag 103 may be fully inflated when the air within inflatable bag is substantially similar to that of the bag volume, such as from 95% to 105% of the bag volume at ambient pressure. When inflatable bag 103 is fully inflated, injected more air into the inflatable bag 103 may increase the pressure within inflatable bag 103 to be greater than the ambient pressure. This may cause inflatable bag 103 to stretch and harden by the increase in bag pressure caused by the injected air. This hardening effect makes the inflatable bag 103 lose its adaptability and pliability to naturally fill the gaps around the user's nose ridge.

Furthermore, inflatable bag 103 may be substantially partially inflated with air when inflatable bag 103 is filled with 1% to 75% of the bag volume at ambient pressure. In embodiments, substantially partial inflation may mean the volume of air in the bag is substantially less than the bag volume at ambient pressure, such as only 75% or 50% of the bag volume at ambient pressure. For example, the inflatable bag 103 that is 50% inflated may be equivalent to 50% fully inflated and 50% not inflated at all and the entire bag may look deflated and flexible and non-uniform. The bag only 50% inflated may look much different from the bag 100% inflated. Accordingly, when inflatable bag 103 is substantially partially inflated at 50%, 50% more air may be injected into inflatable bag 103 before the air pressure within inflatable bag 103 starts to be greater than the ambient air pressure. When inflatable bag 103 is substantially partially inflated, portions of the bag may not be inflated at all. The bag flexibility created by the partial inflation may allow the bag to thicken more at the gaps around the nose ridge but less at the cheeks when the mask is worn. In this case, the formed seal may adapt to the face of a user naturally.

The increase in thickness of mask 100 along the lateral axis of mask 100 may form a seal, border, restriction, etc. along the lateral axis of mask 100 below an upper surface of mask 100 without forming a seal on outer edges 110, 120 or the lower edge of filtering surface 101. This may divert aerosol particles emitted from the user's nose and mouth to the first outer edge 110 and the second outer edge 120 of the filtering surface 101, restricting or limiting the aerosol particles from interacting with a user's eyes or glasses, reducing fogging of glasses. In further embodiments, inflatable bag 103 may have a profile 130 that is contoured to a user's face and include cutouts, projections, etc. The profile 130 of inflatable bag 103 may conform with a user's nose, cheek bones, etc.

In embodiments, filtering surface 101 may be formed of multiple layers, surfaces, etc. For example, filtering surface 101 may have an external layer that is formed of a protective surface, a filtering layer formed of a filtering material, an embedded sealing layer formed of the inflatable bag 103, and an internal layer, wherein the internal layer may be configured to provide comfort of mask 100 and may be formed of a different material than that of the external layer and/or filtering layer. Accordingly, the embedded sealing layer formed of the inflatable bag 103 may be positioned between the filtering layer and the internal layer, wherein the thickness of mask 100 may vary based on the inflation of inflatable bag 103 and the positioning of the air within inflatable bag. When the inflatable bag 103 is partially inflated to fill the gap, mask 100 may passively control a flow of air around the seal towards the first outer edge 110 or the second outer edge 120, wherein the gap is typically formed between the inner surface of the filtering surface 101 and a user's face without the inflatable bag 103.

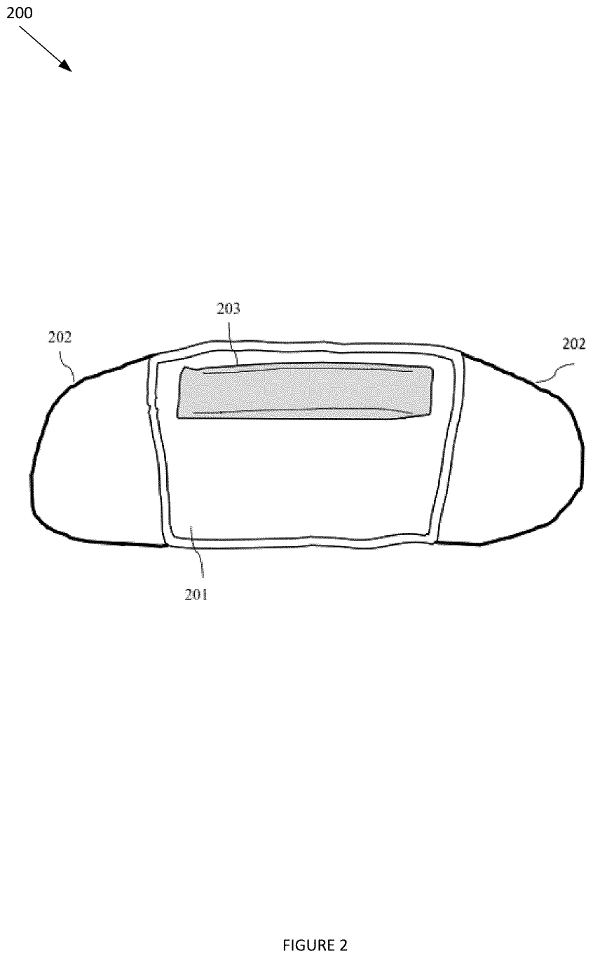

FIG. 2 depicts a mask 200, according to an embodiment. Elements depicted in FIG. 2 may be described above, and for the sake of brevity a detailed description of these elements may be omitted. Mask 200 may include ear straps 202, filtering surface 201, and inflatable bag 203.

Inflatable bag 203 that has a larger volume than a gap may have varying thicknesses at locations corresponding to the gap shapes around the nose ridge. Further, the thickness of inflatable bag 203 may be wider than the gap between the inner surface of the filtering surface and the user's face when inflatable bag 203 is fully inflated.

Inflatable bag 203 may be configured to be positioned on an inner and upper side of filtering surface 201, and be configured to be partially filled with air. Inflatable bag 203 may have a bag volume that is substantially larger than that is needed to fill the nose gaps, such that only partial inflation of inflatable bag 203 is required. Inflatable bag 203 may be configured to form a seal along the inner and upper surface of filtering surface 201. This may passively control the flow of air emitted from the user to outer edges of filtering surface 201.

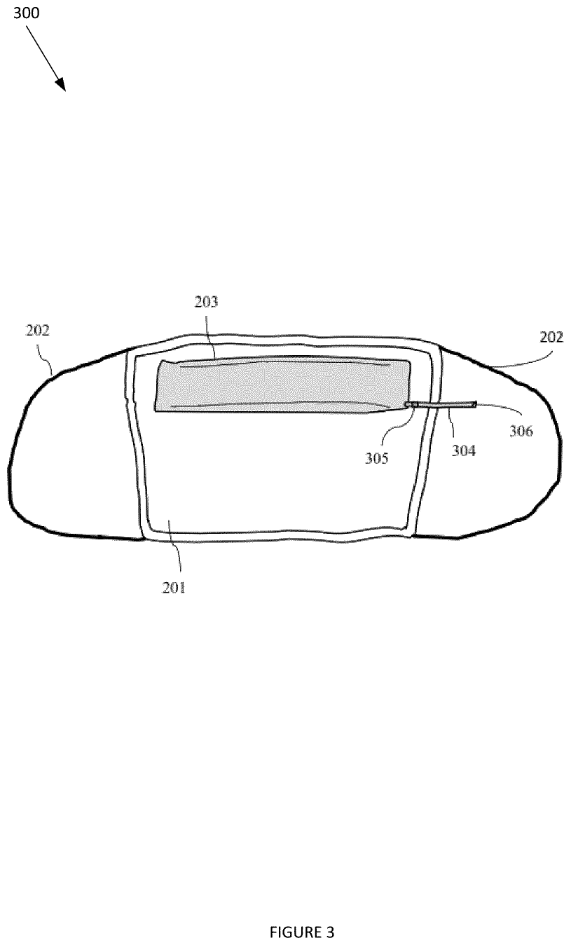

FIG. 3 depicts a mask 300, according to an embodiment. Elements depicted in FIG. 3 may be described above, and for the sake of brevity a detailed description of these elements may be omitted. Mask 300 may include ear straps 202, filtering surface 201, inflatable bag 203, conduit 304 with opening 306, and valve 305.

Conduit 304 may be a tube, pipe, channel with a proximate end positioned within inflatable bag 203 and a distal end opening 306 positioned outside of inflatable bag 203. Utilizing conduit 304, a user may inflate bag 203 while mask 300 is positioned over a user's face or before the user positions the mask over the user's face.

Conduit 304 may also include a valve 305, which is configured to control the air flow in and out of inflatable bag 203. For example, valve 305 may be a one way check valve enabling a user to inflate bag 203 without air escaping bag 203 through conduit 304. In embodiments, a length of conduit 304 may be greater than a distance from the border of inflatable bag 203 to an outer edge of filtering surface 201.

In further embodiments, valve 304 may be formed by twisting or folding conduit 304 to close the air passage in the conduit 304 after the inflatable bag 203 is inflated enough.

Furthermore, a layer of fabric may be placed on top of the inflatable bag and between the bag and its user's face to provide comfort.

In further embodiments, conduit 304 may be positioned on a lower portion of inflatable bag 203, on a central axis of filtering surface 201, and align with a user's mouth. This may more easily allow the user to inflate bag 203 if mask is being worn.

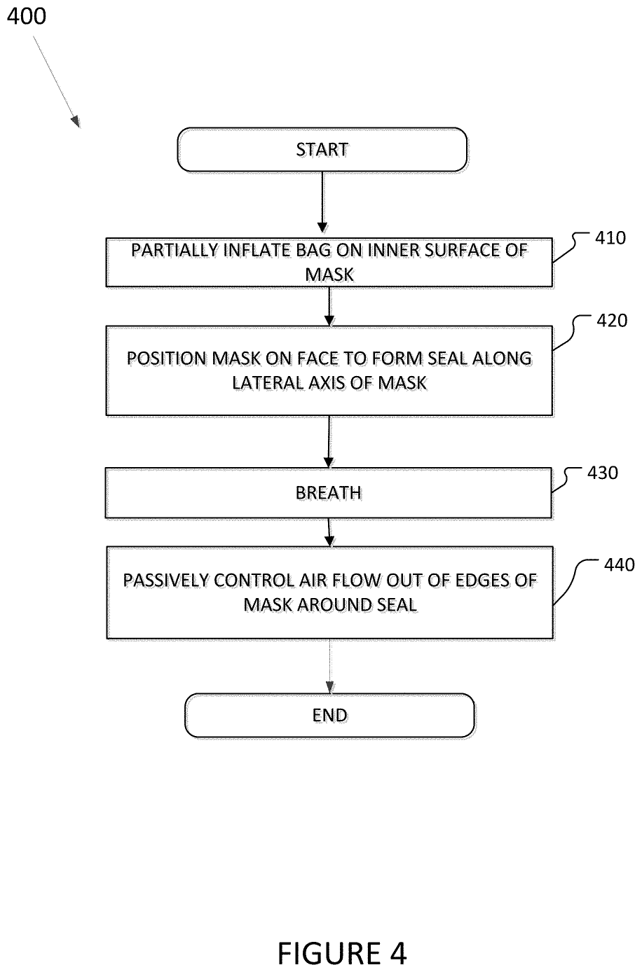

FIG. 4 depicts a method 400 for utilizing a mask, according to an embodiment. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

At operation 410, an inflatable bag extending along a lateral axis of a mask may be partially inflated. The partial inflation of the bag may naturally increase a thickness of the mask being worn at portions aligned with the inflatable bag where otherwise gaps may form. The partial inflation may allow one bag and with one configuration of bag inflation to conform with more sizes and shapes of nose ridges. In order to fully utilize the conformation capability of the partial inflation of a bag, the bag may be made of non-elastic material, such as plastic film, rather than elastic material such as latex.

At operation 420, a wearer may position the mask over his nose and mouth, wherein the inflatable bag is aligned with the ridge of the user's nose. Responsive to positioning the mask over his nose and mouth, a seal may be formed between the user's face and the inflatable bag.

At operation 430, the user of the mask may breath.

At operation 440, the mask may passively control air flow between the user's face and the mask via the inflatable bag. More specifically, the inflatable bag may restrict air from flowing directly upward into the user's eyes and the air may be diverted out of the outer edge of the mask.

Reference throughout this specification to “one embodiment”, “an embodiment”, “one example” or “an example” means that a particular feature, structure or characteristic described in connection with the embodiment or example is included in at least one embodiment of the present invention. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A mask comprising:
a filtering surface configured to filter aerosol particles, the filtering surface including a lateral axis that extends from a first outer edge to a second outer edge;
a first ear strap coupled to the first outer edge;
a second ear strap coupled to the second outer edge;
an inflatable bag positioned on an inner surface of the filtering surface, the inflatable bag being configured to form a seal and passively control a flow of air around the seal towards the first outer edge or the second outer edge.

2. The mask of claim 1, further comprising:
a gap having a gap volume and a varying profile;
a bag volume associated with the inflatable bag, wherein the inflatable bag is filled with air substantially equal to the gap volume, wherein the inflatable bag is configured to be partially inflated before being worn, wherein when the inflatable bag is partially inflated a pressure within the inflatable bag is substantially similar to that of ambient air pressure.

3. The mask of claim 1, wherein when the inflatable bag is partially inflated the inflatable bag is filled up to seventy five percent capacity, wherein air up to one hundred percent capacity is injected into the inflatable bag before the pressure within the inflatable bag is greater than the ambient air pressure.

4. The mask of claim 3, wherein the inflatable bag is positioned below an upper edge of the filtering surface, the filtering surface including an external layer that is formed of a protective surface, a filtering layer formed of a filtering material, an embedded sealing layer formed of the inflatable bag, and an internal layer.

5. The mask of claim 4, wherein the inflatable bag is configured to increase a thickness of the mask at locations proximate to the upper edge of the filtering surface.

6. The mask of claim 5, wherein the inflatable bag has contours that correspond to a nose of a wearer.

7. The mask of claim 1, further comprising:
a conduit with a proximal end positioned within the inflatable bag and an open distal end;
a check valve positioned within the conduit configured to allow one way air flow from the open distal end into the inflatable bag.

8. The mask of claim 7, wherein the conduit is configured to allow the inflatable bag to be inflated when the mask is being worn.

9. The mask of claim 1, wherein the seal diverts the flow of air along the lateral axis of the filtering surface.

10. The mask of claim 9, wherein the flow of air cannot flow along a central axis of the filtering surface through the seal.

11. A method associated with a mask comprising:
coupling a first ear strap to a first outer edge of a filtering surface;
coupling a second ear strap to a second outer edge of the filtering surface;
filtering aerosol particles flowing across the filtering surface, the filtering surface including a lateral axis that extends from the first outer edge to the second outer edge;
inflating an inflatable bag positioned on an inner surface of the filtering surface;
forming a seal with the inflated inflatable bag to
passively control a flow of air around the seal towards the first outer edge or the second outer edge.

12. The method of claim 11, wherein the inflatable bag is configured to be partially inflated before being worn, wherein when the inflatable bag is partially inflated a pressure within the inflatable bag is substantially similar to that of ambient air pressure, wherein a gap has a gap volume and a varying profile, and the inflatable bag has a bag volume associated with the inflatable bag, wherein partially inflating the inflatable bag inflates the inflatable bag with air substantially equal to the gap volume.

13. The method of claim 11, wherein when the inflatable bag is partially inflated the inflatable bag is filled up to seventy five percent capacity, wherein air up to one hundred percent capacity may be injected into the inflatable bag before the pressure within the inflatable bag is greater than the ambient air pressure.

14. The method of claim 11, wherein the inflatable bag is positioned below an upper edge of the filtering surface, the filtering surface including an external layer that is formed of a protective surface, a filtering layer formed of a filtering material, an embedded sealing layer formed of the inflatable bag, and an internal layer.

15. The method of claim 14, further comprising:
increasing a thickness of the mask, via the inflatable bag, at locations proximate to the upper edge of the filtering surface.

16. The method of claim 15, wherein the inflatable bag has contours that correspond to a nose of a wearer.

17. The method of claim 11, further comprising:
positioning a conduit with a proximal end positioned within the inflatable bag;
controlling air flow through the conduit via a check valve positioned within the conduit.

18. The method of claim 17, further comprising:
inflating the inflatable bag to be inflated when the mask is being worn.

19. The method of claim 11, further comprising:
diverting the flow of air along the lateral axis of the filtering surface.

20. The method of claim 19, wherein the flow of air cannot flow along a central axis of the filtering surface through the seal.
